(19) United States
(12) Patent Application Publication
(10) Pub. No.: US 2015/0005801 A1
(43) Pub. Date: Jan. 1, 2015

(54) MICROCATHETER SYSTEM

(71) Applicant: Covidien LP, Mansfield, MA (US)

(72) Inventors: James Marquis, Newport Beach, CA (US); Eric Rowson, Laguna Niguel, CA (US)

(73) Assignee: Covidien LP, Mansfield, MA (US)

(21) Appl. No.: 13/928,638

(22) Filed: Jun. 27, 2013

(51) Int. Cl. A61M 25/10; A61F 2/01; A61M 25/00

(52) U.S. Cl. CPC A61M 25/10; A61M 25/0097; A61F 2/01; A61M 2025/0004; A61F 2002/011
USPC 606/194

(57) ABSTRACT

A system for use in a vascular procedure includes a guidewire dimensioned to remotely access a neurovascular space, a microcatheter for positioning over the guidewire, an interventional treatment element for passage within the catheter member of the microcatheter, and an outer guide positionable over the catheter member of the microcatheter upon removal of the catheter hub. The microcatheter includes an elongated catheter member and a catheter hub. The catheter member defines a longitudinal axis and has a longitudinal length and with proximal and distal ends. The catheter hub is connected to the proximal end of the catheter member and is dimensioned and adapted to be selectively released from the catheter member. The outer guide is advanceable over the catheter member of the microcatheter after the catheter hub is released from the catheter member to a location proximate the lesion. The interventional treatment element is adapted to perform treatment on the lesion within the vasculature

### BACKGROUND

1. Technical Field

The present disclosure relates generally to medical systems and methods and, more particularly, relates to a microcatheter system and associated methodology for accessing, diagnosing, or treating conditions in blood vessels, such as blood vessels within remote neurovasculature. The present disclosure further relates to a microcatheter system incorporating a microcatheter with a catheter hub which is detachable for facilitating introduction of other intravascular treatment devices for, e.g., removing obstructive material or introducing a therapeutic agent, within the neurovascular space.

2. Description of Related Art

Microcatheters are commonly employed to access vascular treatment sites or deliver interventional medical devices in the vasculature. The column support of these microcatheters often is insufficient to navigate through the distal reaches of the neurovasculature thereby necessitating the use of a guide catheter to act as a conduit to help support microcatheter access. The use of the guide catheter increases the time to perform the procedure such as accessing a clot and restoring blood flow resulting from ischemic stroke. Newer distal access guide catheters have been developed which are slightly longer, thinner, a bit more flexible than early generations, but are still deficient in consistently penetrating an occlusion and providing timely access to treatment sites.

### SUMMARY

Accordingly, the present disclosure is directed to a microcatheter capable of navigating over a guidewire into remote vasculature without requiring a guide support. In one aspect, the microcatheter is intended for use in the neurovasculature and is advanced from the groin area over the aortic arch and into the cerebral vasculature without requiring a guide catheter for support while travelling over the aortic arch. The microcatheter may be capable of crossing a lesion followed by, e.g., deployment of a stent across the lesion, instantly restoring blood flow. The microcatheter may have a removable catheter hub, which, upon removal, permits tracking of a larger catheter or outer guide over the microcatheter to a location in the neurovasculature sufficient to perforin aspiration for clot retrieval and/or another surgical procedure.

In one embodiment, a system for use in a vascular procedure includes a guidewire dimensioned to remotely access a neurovascular space, a microcatheter having an elongate catheter member and a catheter hub, an interventional treatment element for passage within the catheter member of the microcatheter, and an outer guide positionable over the catheter member of the microcatheter upon removal of the catheter hub. The catheter member defines a longitudinal axis and has a longitudinal length, and proximal and distal ends. The catheter member comprises a material exhibiting sufficient flexibility to traverse remote locations in the vasculature and sufficient strength to permit transmission of torque along the longitudinal length of the catheter member and to pass through a lesion in the vasculature in the absence of an independent additional outer guide. The catheter hub is connected to the proximal end of the catheter member and is dimensioned and adapted to be selectively released from the catheter member. The outer guide is advanceable over the catheter member of the microcatheter after the catheter hub is released from the catheter member to a location proximate the lesion. The catheter hub is releasably connected to the proximal end of the catheter member through one of thread means, a bayonet coupling, a snap fit mechanism, an interference fit, an adhesive or a chemical bond.

The interventional treatment element is adapted to perform treatment on a malformation or the lesion within the vasculature. In embodiments, the interventional treatment element is dimensioned to be introduced within the catheter member of the microcatheter upon removal of the guidewire. The interventional treatment element may be selected from the group consisting of a stent, a coil, a flow diverter, a flow restoration element, a thrombectomy element, a retrieval element, an aspirator and a snare. In the alternative, the interventional treatment element may be a liquid embolic system.

The outer guide may be a balloon catheter. The balloon catheter is dimensioned to receive the interventional treatment device during withdrawal thereof subsequent to performing the treatment on the lesion. The balloon catheter may be connectable to an aspirator to facilitate removal of materials during the procedure.

The system may include an extension member which is connectable to the proximal end of the catheter member upon removal of the catheter hub. The outer guide is advanceable over the extension member and the catheter member of the microcatheter. The extension member and the proximal end of the catheter member may include corresponding structure to connect the extension member to the catheter member.

The catheter member of the microcatheter may include a catheter tip segment. The catheter tip segment may comprise a soft material relative to a neighboring segment of the catheter member proximal of the catheter tip segment to minimize trauma during navigation through the vasculature. At least one radiopaque marker may be adjacent the catheter tip segment of the catheter member of the microcatheter.

The catheter tip segment of the catheter member may be in telescoping relation with respect to at least the neighboring segment of the catheter member adjacent the catheter tip segment. The catheter tip segment and the neighboring segment may be adapted for relative longitudinal movement between a first retracted position at least partially enclosed within the neighboring segment of the catheter member and a second extended position exposed beyond the neighboring segment.

The catheter member of the microcatheter may include one of a hypotube segment or reinforced polymer tube segment disposed at least adjacent the proximal end of the catheter member. The catheter member of the microcatheter also may include a braided segment disposed at least adjacent the proximal end of the catheter member.

A method for performing a neurovascular procedure is disclosed. The method includes:

accessing a treatment site within a neurovascular space with a guidewire:

advancing a microcatheter over the guidewire to traverse remote locations in the neurovasculature to access the treatment site and penetrate a lesion located at the treatment site without the assistance of an independent outer guide support positioned about the microcatheter;

removing the guidewire from the microcatheter;

introducing an interventional treatment element through the microcatheter to a location adjacent the lesion within the neurovasculature;

treating the lesion with the interventional treatment element;

removing a catheter hub member from the microcatheter leaving a catheter member of the microcatheter within the neurovasculature;

positioning an outer guide over the catheter member of the microcatheter and advancing the outer guide to a location proximate the treatment site; and

withdrawing the microcatheter through the outer guide.

Treating the lesion may include deploying a flow restoration device within the lesion to restore flow through the adjacent neurovasculature whereby the method may include removing at least a portion of the lesion from the neurovasculature while withdrawing the flow restoration device through the outer guide.

A balloon member of the outer guide may be expanded to secure the outer guide within the neurovasculature. The interventional treatment device may be withdrawn through the outer guide and aspiration supplied through the outer guide. Withdrawing the microcatheter and the interventional treatment device may be performed simultaneously.

Treating the lesion may include delivering the treatment element selected from the group consisting of a stent, a coil, an embolic solution, glue, a flow diverter, a flow restoration element, a thrombectomy element, a retrieval element, an aspirator and a snare.

Advancing the microcatheter may include penetrating the lesion with a catheter tip segment of the microcatheter in which the catheter tip segment is relatively soft relative to a neighboring segment neighboring the catheter tip segment. The method may include introducing a second interventional element within the outer guide subsequent to withdrawing the microcatheter.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 illustrates a side elevation view with portions removed of a microcatheter of the system in accordance with the principles of the present disclosure, including a catheter hub and an elongate catheter extending from the catheter hub;

FIG. 2 is a cross sectional view of the catheter member taken along the lines 2-2 of FIG. 1;

FIG. 3 is a partial side elevation view of the microcatheter illustrating the catheter hub released from the catheter member;

FIGS. 4A-4B illustrate an extension member of the system with FIG. 4A depicting the extension member detached relative to the catheter member and FIG. 4B depicting the extension member mounted to the catheter member upon release of the catheter hub from the catheter member;

FIG. 5A-5B are partial side elevation views illustrating an alternate embodiment for releasably mounting the catheter hub to the catheter with FIG. 5A depicting the catheter hub released from the catheter member and FIG. 5B depicting the catheter hub mounted to the catheter member;

FIG. 6A-6B are partial side elevation views illustrating another alternate embodiment for releasably mounting the catheter hub to the catheter with FIG. 6A depicting the catheter hub released from the catheter member and FIG. 6B depicting the catheter hub mounted to the catheter member;

FIG. 7 is a side elevation view in partial cross-section illustrating another alternate embodiment for releasably mounting the catheter hub to the catheter;

FIG. 8 is a side elevation view in partial cross-section illustrating another alternate embodiment for releasably mounting the catheter hub to the catheter;

FIG. 9 is a side elevation view of an outer guide of the system;

FIG. 10 is a cross-sectional view of the outer guide taken along the lines 10-10 of FIG. 9;

FIG. 11 is a perspective view of a guidewire of the system;

FIGS. 12-16 are views of various interventional devices of the system;

FIG. 17 is a flow chart illustrating an exemplary use of the system in an intravascular procedure;

FIG. 18 is a view illustrating passage of the catheter tip segment of the microcatheter through a lesion within the vasculature;

FIG. 19 is a view illustrating deployment of a stent within the vasculature;

FIG. 20 is a view illustrating deployment of a flow diverter within the vasculature;

FIG. 21 is a view illustrating deployment of a flow restoration device within the vasculature;

FIG. 22 is a view of an alternate embodiment of the microcatheter of the system illustrating a main catheter segment and a retractable catheter tip segment with the catheter tip segment depicted in a first position at least partially disposed within the main catheter segment; and

FIG. 23 is a view of the microcatheter of FIG. 22 illustrating the retractable catheter tip segment depicted in a second position exposed from the main catheter segment.

### DETAILED DESCRIPTION

In the following description, the terms “proximal” and “distal” as used herein refer to the relative position of the instrument in a lumen. The “proximal” or “trailing” end of the instrument is the segment extending outside the body closest to the clinician. The “distal” or “leading” end of the instrument is the remote segment placed into a body lumen from the entrance site.

The system of the present disclosure has particular application in a neurovascular procedure, but may be used in any interventional, diagnostic, and/or therapeutic procedure including coronary vascular, peripheral vascular, and gastro-intestinal applications in addition to neurovascular applications. The system may include a guidewire, a microcatheter, an interventional device introducible within the microcatheter and/or an outer guide which is positionable over the microcatheter. Other instrumentation is also contemplated. In the figures below, the full length of the various instruments of the system may not be shown. The respective lengths of the various instruments can vary depending on the type of interventional procedure.

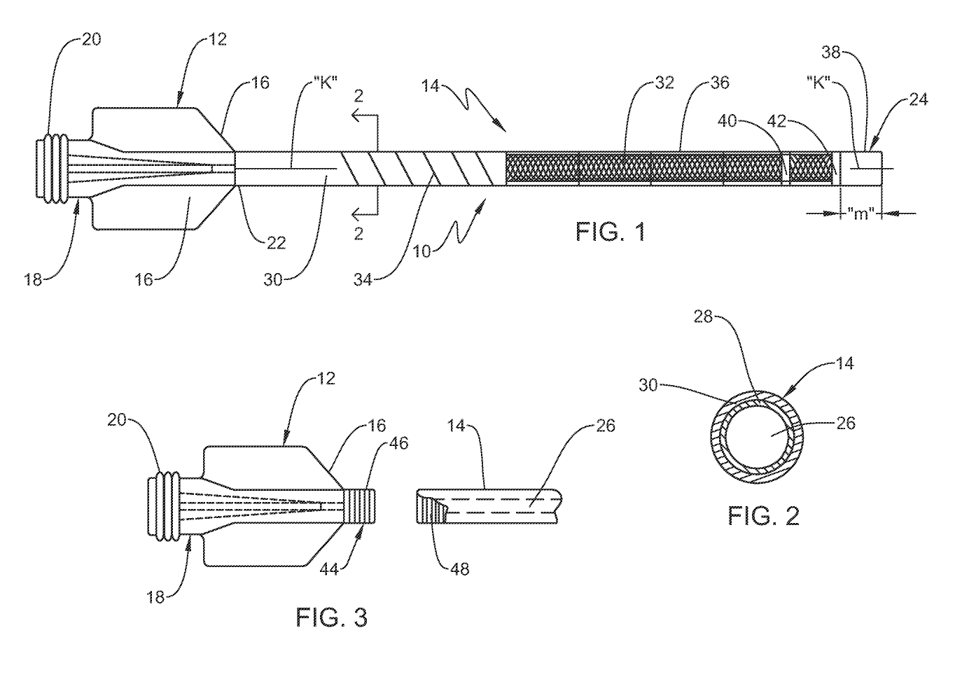

Referring now to FIG. 1, a microcatheter 10 of the system is illustrated. The microcatheter 10 includes a housing or catheter hub 12 and an elongated catheter member 14 extending from the catheter hub 12, and defining a longitudinal axis “k”. The catheter hub 12 may include a pair of opposed manipulative wings 16 to assist in maneuvering the catheter hub 12 and a fitting 18. The fitting 18 has a threaded segment 20 to facilitate attachment to a syringe and/or a vacuum or aspiration source (not shown). The catheter hub 12 further may include a strain relief (not shown) which is positionable over a segment of the catheter member 14. The catheter hub 12 is removably mounted or coupled to the catheter member 14 as will be discussed in greater detail hereinbelow.

The catheter member 14 has proximal and distal ends 22, 24 and defines a longitudinal lumen 26 (shown in FIG. 2) extending the length of the catheter member 14. The catheter member 14 provides sufficient torsional and lateral stiffness to enable steering of the catheter member 14 through the tortuous regions of the vasculature, particularly the distal reaches of the neurovasculature. In embodiments, the catheter member 14 has a stiffness profile and torquability that removes the necessity of a guiding catheter for support to permit navigation of the catheter member 14 through the aortic arch and into the neurovaseulature.

With reference to FIGS. 1-2, the catheter member 14 may include an inner liner 28 which extends along the entire length of the catheter member 14, a hypotube 30 positioned over at least the proximal end segment of the inner liner 28 and a braid 32 positioned over at least the distal end segment of the inner liner 28. The inner liner 28 may be fabricated from polytetrafluoroethylene (PTFE). The hypotube 30 may be fabricated from stainless steel and have a spiral cut pattern 34. The spiral cut pattern 34 may be continuous or discontinuous, and have a variable pitch from the proximal end toward the distal end or variations thereof. In the alternative, the hypotube 30 may include a skive or window pattern. In some embodiments, the hypotube 30 may extend the entire length of the catheter member 14. In other embodiments, a reinforced polymer tube may be substituted for the hypotube 30.

The braid 32 may be fabricated from nitinol and have a continuous pick count, varying pick count, and/or sections with different diameter braid wire. An outer jacket 36 may be positioned over or embedded in the braid 32 and possibly the hypotube 30. The distal end of the braid 32 starts a predetermined distance “m” from the distal end 24 of the catheter member 14 leaving a catheter tip segment 38 devoid of a braid 32. The catheter tip segment 38 is soft relative to the remainder of the catheter member 14 to minimize the potential of trauma to the vasculature. In embodiments, the catheter tip segment 38 may comprise only the outer jacket 36 and/or the inner liner 28. In embodiments, the catheter tip segment 38 may be shaped having a predefined curved or bent profile. Proximal and distal marker bands 40, 42 may be embodied within the catheter member 14 adjacent the catheter tip segment 38 to assist in visualization of the catheter member 14 during the interventional procedure.

The length and diameter of the catheter member 14 may vary depending on the particular application. In a neurovascular application, the length may range from about 90 centimeters to about 180 centimeters, and the inner diameter of the catheter member may range from about 0.0165 inches to about 0.027 inches. Other dimensions are also contemplated.

Other arrangements for the elongated catheter member 14 are also envisioned. For example, the catheter member 14 may include some of the structural features of the commercially available microcatheters such as the Echelon™, Marathon™, and Nautica™ microcatheters sold by Covidien LP, Irvine, Calif.

Referring now to FIG. 3, in conjunction with FIG. 2, the releasable coupling of the catheter hub 12 to the catheter member 14 will be discussed. In one embodiment, the catheter hub 12 includes a mounting collar 44 having an external thread 46 and the catheter member 14 includes an internal thread 48. The internal thread 48 may be within the inner liner 28 and/or the hypotube 30. The threads 46, 48 are dimensioned to threadably engage each other to either secure or release the catheter hub 12 relative to the catheter member 14. More particularly, subsequent to placement of the elongated catheter 14 within the intravascular site, the catheter hub 12 may be removed from the catheter member 14 by simply rotating the catheter hub 12 about the longitudinal axis “k” and maintaining the catheter member 14 in a stationary condition. Other arrangements for releasably securing the catheter hub 12 relative to the catheter member 14 are also envisioned.

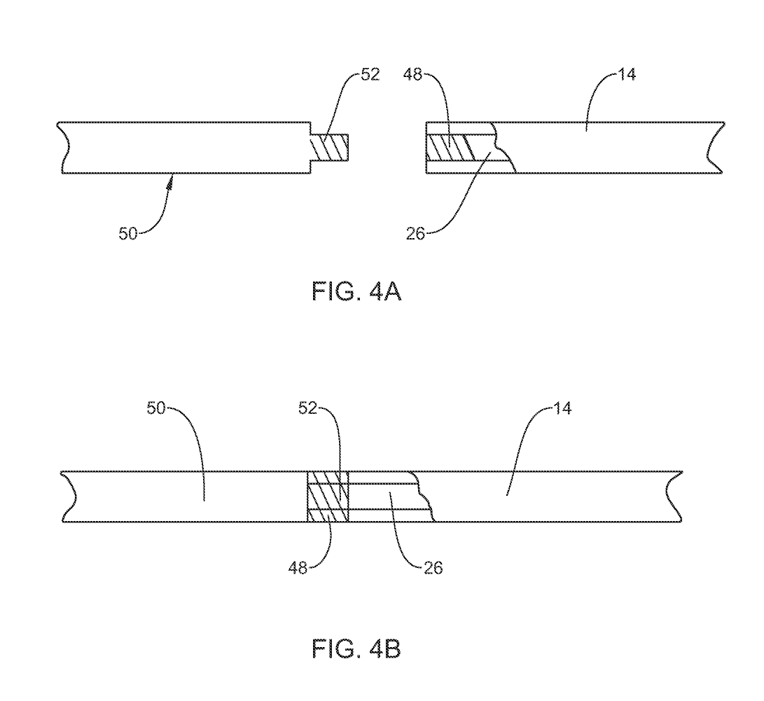

FIGS. 4A-4B illustrate an extension member 50 which may be an optional component of the system 10. The extension member 50 is connectable to the elongate catheter member 14 subsequent to removal thereof from the catheter hub 12. The extension member 50 extends the effective length of the catheter member 14, which may be necessary depending on the vascular procedure to be performed. For example, in a neurovascular procedure, there may be a need to extend the length of the microcatheter 10 if the microcatheter is not of “exchange catheter” length. In embodiments, the extension member includes a threaded extension collar 52, which threadably engages the internal thread 48 of the catheter member 14 in a similar manner to the mounting collar 44 of the catheter hub 12. The extension member 50 and the catheter member 14 may include any of the releasable coupling mechanisms for coupling the catheter hub 12 to the catheter member 14 discussed hereinbelow. The extension member 50 may be fabricated from any material suitable for a medical catheter. The extension member 50 may define an outer diameter approximating the outer diameter of the catheter member 14. The extension member 50 may define a length ranging from about 30 centimeters to about 150 centimeters or more. Further details of the use of the extension member 50 will be discussed in greater detail hereinbelow.

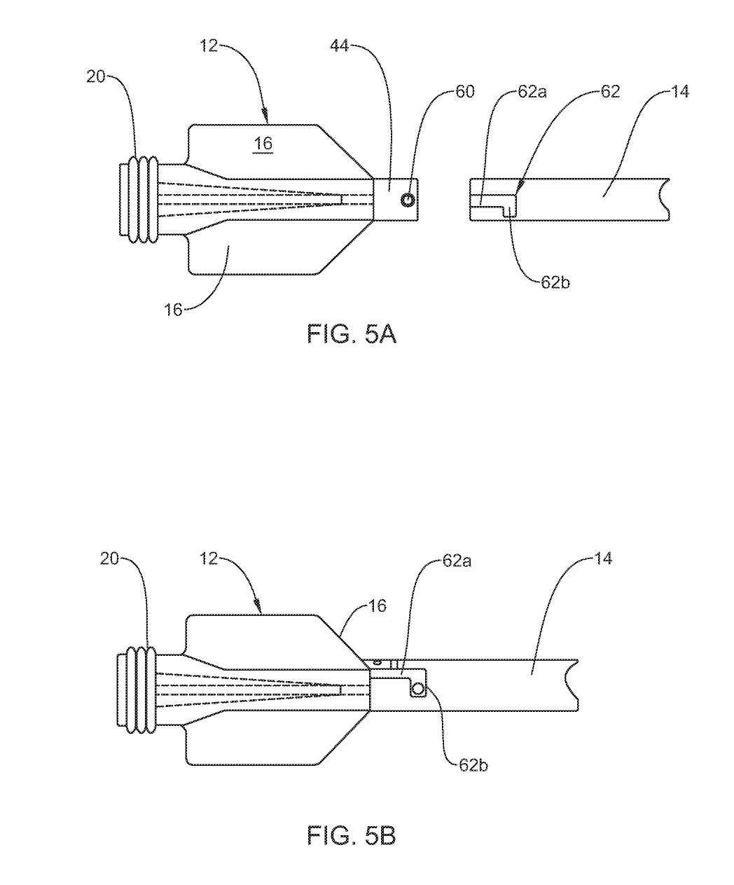

With reference to FIGS. 5A-5B, an alternate embodiment for releasably coupling the catheter hub 12 to the catheter member 14 is illustrated. The microcatheter 10 may include a bayonet mount or coupling for releasably coupling the catheter hub 12 to the catheter member 14. With this arrangement, the mounting collar 44 of the catheter hub 12 includes at least one external pin 60 extending radially outwardly from the mounting collar 44. The elongated catheter member 14 includes a corresponding L-shaped slot 62. To couple the catheter hub 12 to the catheter member 14, the pin 60 is aligned with the receiving leg 62a of the slot 62, and the catheter hub 12 and the catheter member 14 are moved toward each other. Once the pin 60 is aligned with the locking leg 62b of the slot 62, the catheter hub 12 is rotated relative to the catheter member 14 to secure the pin 60 within the locking leg 62b as shown in FIG. 5B. The pin 60 and the locking leg 62b may be dimensioned to establish a frictional relation between the components. The bayonet coupling may include a pair of diametrically opposed pins 60 and a pair of corresponding of L-shaped slots 62 defined within the catheter member 14.

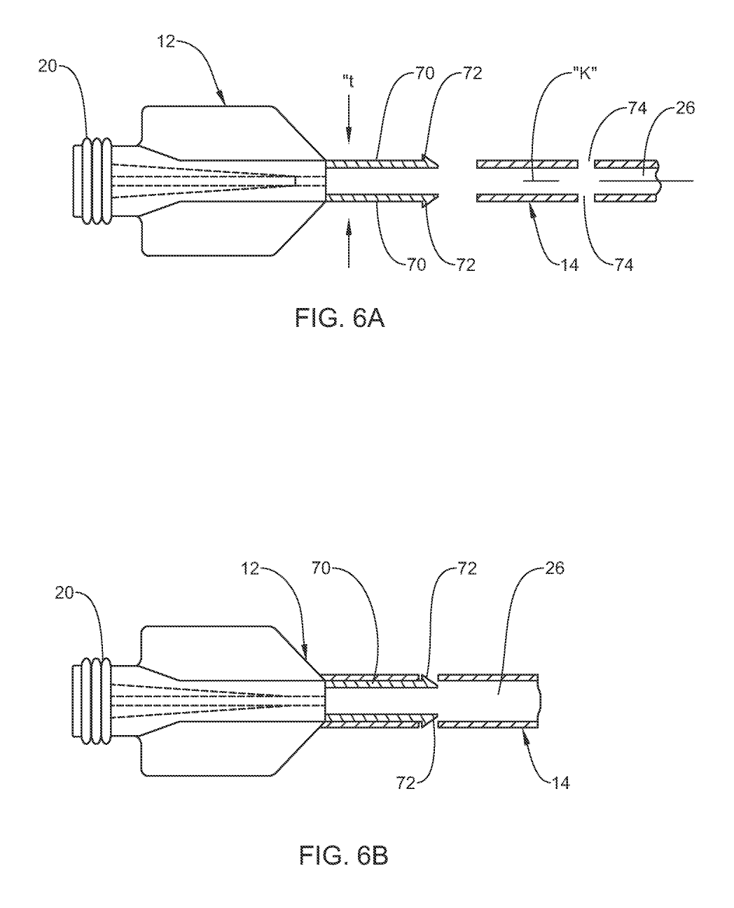

FIGS. 6A-6B illustrate another arrangement for releasably coupling the catheter hub 12 to the catheter member 14. In accordance with this embodiment, the catheter hub 12 includes a pair of resilient locking legs 70 which extend outwardly along the longitudinal axis “k”. The locking legs 70 are normally biased to the condition shown in FIG. 5A and include radially outwardly extending locking detents 72. The catheter member 14 includes a pair of opposed locking slots 74. In one embodiment, the locking slots 74 extend through the outer wall of the catheter member 14. To couple the components, the locking detents 72 may be displaced toward each other by exertion of a radial inward force along the direction of directional arrows “t”, and introducing the locking detents 72 within the lumen 26 of the catheter member 14. The locking legs 70 are aligned with the locking slots 74 of the catheter member 14, and released whereby the locking detents 72 are received within the locking slots 74 in secured relation therewith. To decouple the catheter hub 12 from the catheter member 14, the locking detents 72 may be compressed or displaced radially inwardly by exerting a force on the external surface of the locking detents. Once the locking detents 72 clear the locking slots 74, the catheter hub 12 may be removed from the catheter member 14.

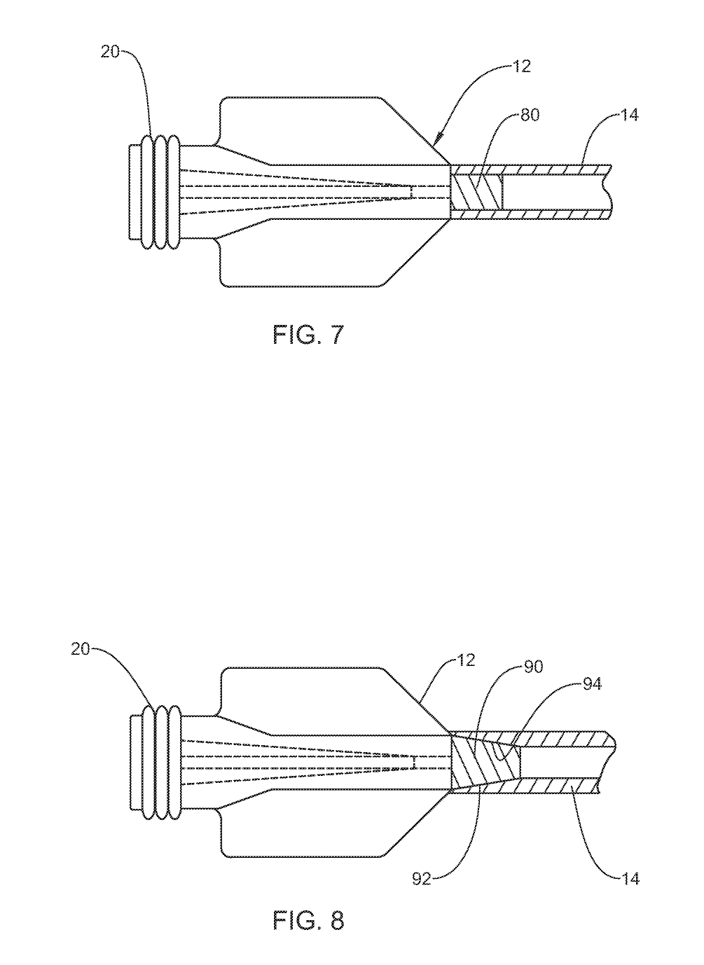

FIG. 7 illustrates another embodiment for releasably coupling the catheter hub 12 and the catheter member 14. In accordance with this embodiment, the catheter hub 12 includes a coupling member 80 extending along the longitudinal axis at its distal end. The coupling member 80 is received within the lumen 26 of the catheter member 14, and is corresponding dimensioned to establish an interference fit with the internal surface of the catheter member 14. In embodiments, the coupling member 80 defines a cross-sectional dimension or diameter at least equal to, or slightly greater than, the cross-sectional dimension or diameter of the lumen 26 of the catheter member 14. The tolerances or dimensions of the components may be selected to provide controlled release of the catheter hub 12 at a predetermined release force. In other embodiments, e.g., depicted in FIG. 8, the coupling member 90 and the internal surface of the catheter member 12 may include cooperating respective tapered surfaces 92, 94 to establish a Morse taper providing a more secured coupling relation of the components.

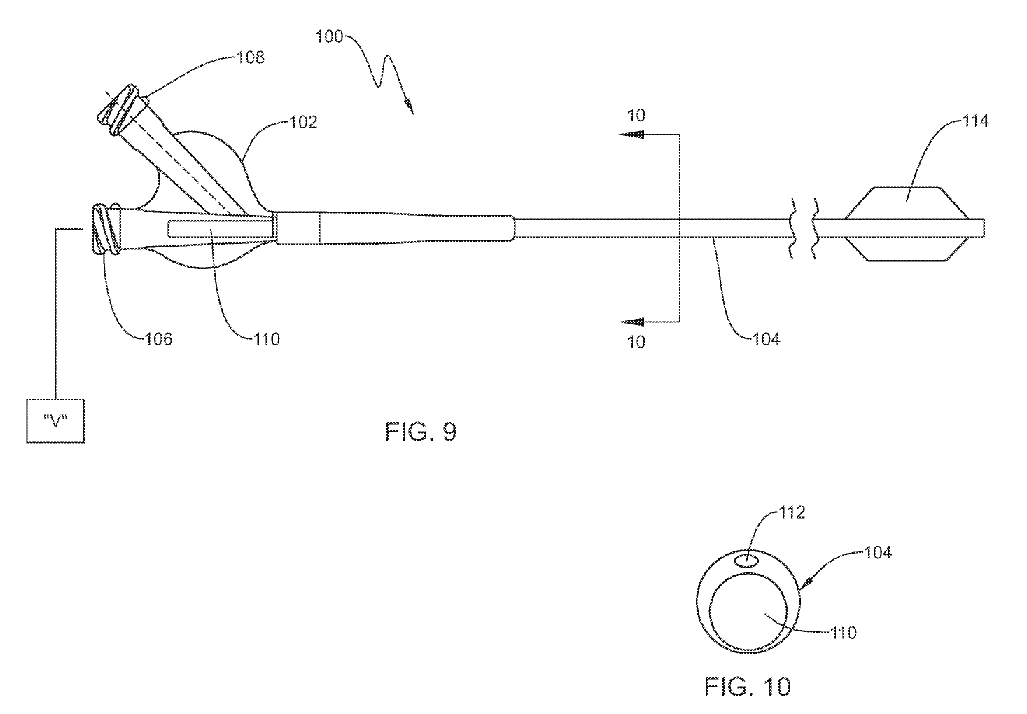

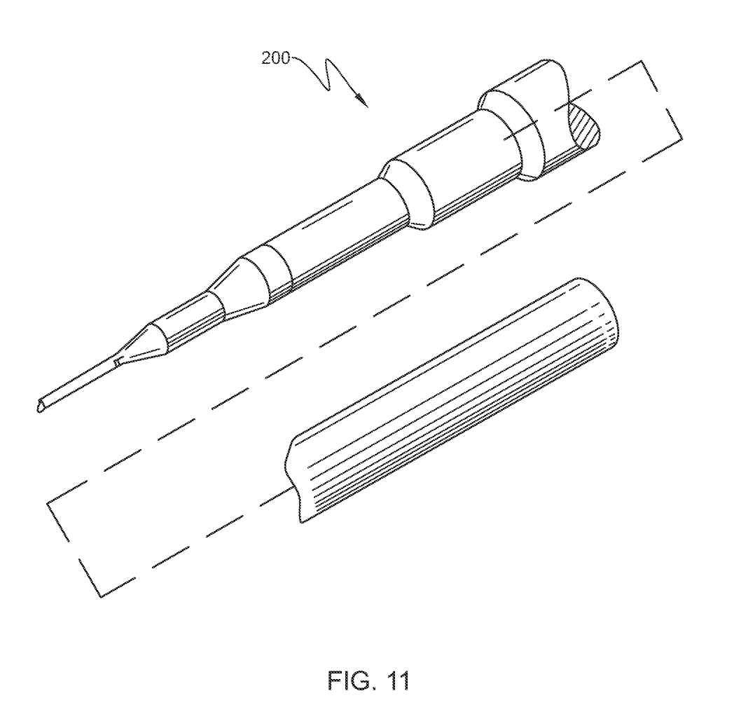

Referring now to FIGS. 9-10, an outer guide 100 of the system will be discussed. The outer guide 100 may be any catheter adapted for intravascular use. The outer guide 100 may include a housing 102 and a guide member 104 extending from the housing 102. The housing 102 may include a first port 106 for connection to a syringe, aspiration device (shown schematically as “v”) and/or for the introduction of instrumentation, and a second inflation port 108. The guide member 104 is connected to the housing 102 and defines first and second lumens 110, 112 in fluid communication with the first and second ports 106, 108. An expandable balloon 114 is mounted to the guide member 104 adjacent the distal end. The balloon 114 is selectively expandable through introduction of fluids through the second inflation port 108, which communicate through the second lumen 112 of the guide member 104. An opening (not shown) extends through the wall of the guide member 104 within the interior of the balloon 114 to permit the inflation fluids to enter the balloon volume. The first lumen 110 may be in fluid communication with the aspiration device “v”. The diameter of the first lumen 110 is greater than the outer diameter or dimension of the catheter member 104 of the microcatheter 10 to enable the guide member to track over the catheter member 14 upon removal of the catheter hub 12. In a neurovascular procedure, the diameter ranges from about 0.017 inches to about 0.030 inches or more. The wall of the guide member 104 has sufficient lateral support to permit introduction of instrumentation through the first lumen 110 for performing an interventional procedure. In embodiments, the outer guide 100 may be replaced with a catheter devoid of a balloon.

FIG. 11 illustrates the guidewire 200 of the system. The guidewire may be any conventional guidewire. In accordance with one application of the present disclosure, the maximum outer diameter of the guidewire ranges from about 0.008 inches to about 0.018 inches. These diameters are standard for guidewires used, e.g., in a neurovascular procedure. Other diameters are contemplated for cardiovascular, peripheral vascular, and gastrointestinal applications. The diameter of the guidewire may remain relatively constant over a major portion of the length of the guidewire. In the alternative, the leading or distal end incorporates a generally tapered or narrowed configuration to permit flexure while navigating the tortuous vasculature. The guidewire 200 may include a number of tapered segments which may or may not be continuous. The length of the guidewire may range from about 30 to about 400 centimeters. Other lengths are also contemplated.

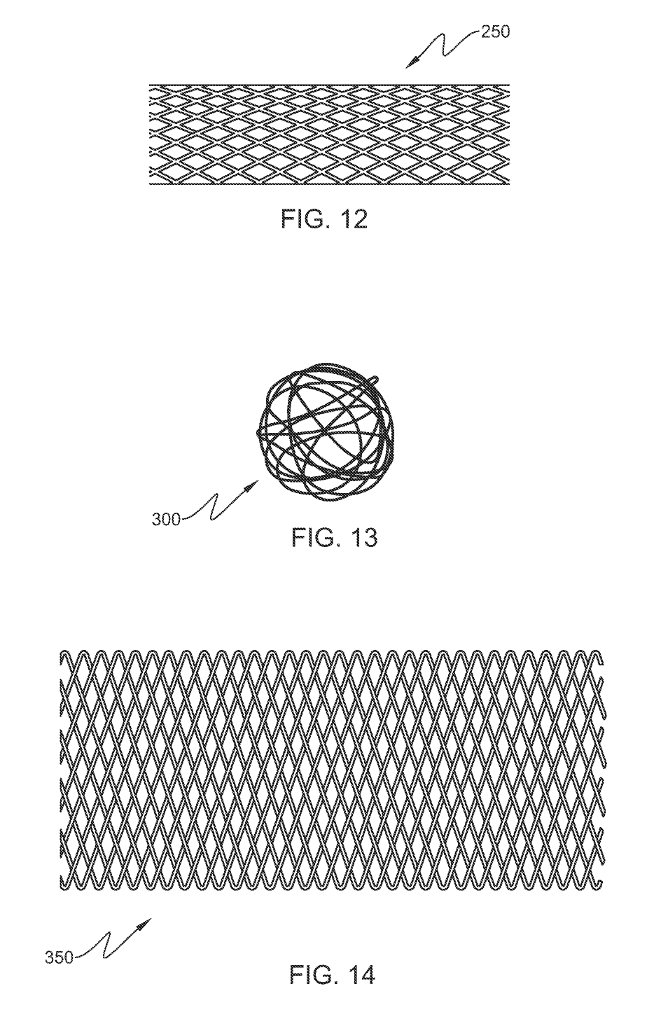

FIGS. 12-16 illustrate various exemplary interventional devices or elements incorporated within the system of the present disclosure. FIG. 12 illustrates an intravascular stent 250 which may be self-expanding or balloon expandable. The intravascular stent may be fabricated from a nickel-titanium alloy (Nitinol) and generally define a lattice structure. One suitable peripheral vascular stent is the commercially available ProtégéRX™ stent sold by Covidien LP, Plymouth, Minn. The ProtégéRX™ stent is deployable to achieve its predetermined diameter to exert a gentle outward force to establish patency of the vessel.

FIG. 13 illustrates an embolic coil 300 which is utilized in endovascular treatment of aneurysms and arteriovenous malformations (AVMs). A plurality of coils may be advanced through a catheter into the affected area of the neurovasculature, filling the weakened portion of the vessel. Once in place, the body responds by forming a clot around the coil, further reducing the pressure and risk of rupture. One suitable coil is the commercially available Axium™ coil sold by Covidien LP, Irvine, Calif.

FIG. 14 illustrates a flow diverter 350 which may be a component of the system. Flow diversion is a technique used primarily to treat wide-necked neurovascular aneurysms in which the device is placed in the parent blood vessel rather than in the aneurysm sac. One example of a flow diverter is the Pipeline® device sold by Covidien LP, Irvine, Calif., which restores original, natural blood circulation while providing permanent long-term occlusion. During the procedure, the flow diverter 350 in, e.g., the form of a braided cylindrical scaffolding mesh, is implanted across the aneurysm neck. The flow diverter 350 may self expand to engage the vessel wall. This slows or diverts the flow of blood into the aneurysm, which allows for the diseased vessel to heal.

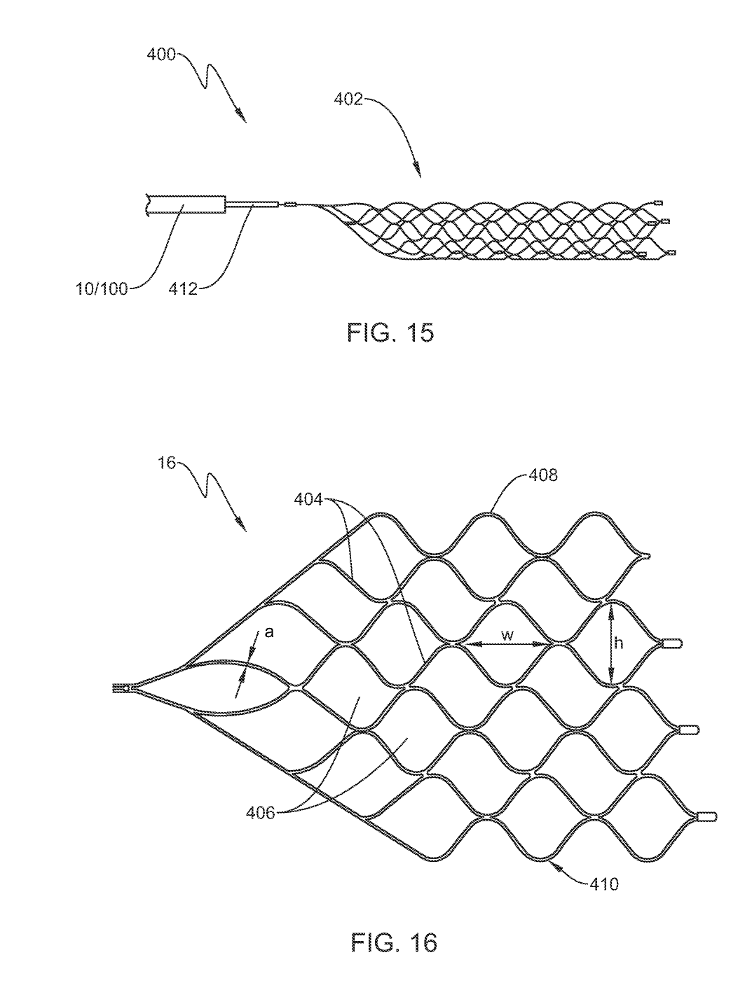

FIGS. 15-16 illustrate a flow restoration device 400 for incorporation within the system of the present disclosure. One flow restoration device may be the Solitaire™ FR revascularization device which is a mechanical thrombectomy device sold by Covidien LP, Irvine, Calif. Details of the SOLITAIRE™ device are described in commonly assigned U.S. Patent Publication No. 2012/0083868, the entire contents of which is hereby incorporated by reference herein. The thrombectomy device is adapted to restore blood flow and retrieve clot in patients experiencing acute ischemic stroke. The SOLITAIRE™ device is a stent based design deployable within a clot, and adapted to engage and remove the clot upon retrieval of the flow restoration device. In embodiments, a self-expanding member or capturing element 402 of the SOLITAIRE™ device is deployed to expand relative to a clot or lesion, and may expand to encompass the lesion. The self-expanding member 402 may include a plurality of individual filaments 404 and individual cells 406, as well as a first edge 408 and a second edge 410. The first edge 408 and second edge 410 can be formed, for example, from cutting a preformed, etched tube longitudinally along the length of the tube. (In FIG. 16, the self-expanding member 402 is shown in an unrolled, open state). The self-expanding member 402 can be curled such that edges 408 and 410 overlap one another when the self-expanding member 402 is in a volume-reduced form. While in a volume-reduced form, the self-expanding member 402, similar to a wire mesh roll, or piece of paper, can be curled up such that it can be introduced into a microcatheter and moved within the microcatheter. The self-expanding member 402 can have a central longitudinal axis while in both a volume-reduce form and when fully or partially expanded. Upon release from the microcatheter 10 or outer guide 100, the curled-up self-expanding member 402 can spring open and attempt to assume a fully expanded shape. Upon expansion, the self-expanding member 402 can expand towards an inner wall of a vessel, or towards a thrombus occluding the inner wall of a vessel. The extent of any overlap of the self-expanding member 402 within the vessel after expansion can be governed by the vessel size. For example, in narrower vessels a greater overlap of the edges 408 and 410 can occur, whereas in wider vessels the overlap can be smaller, or even an “underlap” may occur, in which case the edges 408 and 410 are separated by an open gap or space within the vessel.

A deployment/retrieval rod or guidewire 412 may be connected to the capturing element 402 to permit deployment of the self-expanding member 402 from the microcatheter 10 or outer guide 100, and retrieval subsequent to capturing or snaring the lesion through, e.g., the microcatheter 10. For example, the retrieval rod 412 and the self-expanding member 402 may be retracted to remove the clot or lesion from the vessel area or through an extraction device, such as the microcatheter 10 or outer guide 100, thus reopening the blocked vessel. The SOLITAIRE™ device has proven to be highly effective in removing clots in stroke patients in a reduced operative time.

Other treatment elements envisioned within the system include the introduction of liquid embolics. The liquid embolic material may be injected through the microcatheter 10 into the effected area of the brain, where it begins to solidify, reducing the flow of blood to the aneurysm and therefore the likelihood of rupture. One suitable liquid embolic is the commercially available Onyx® LES sold by Covidien LP, Irvine, Calif.

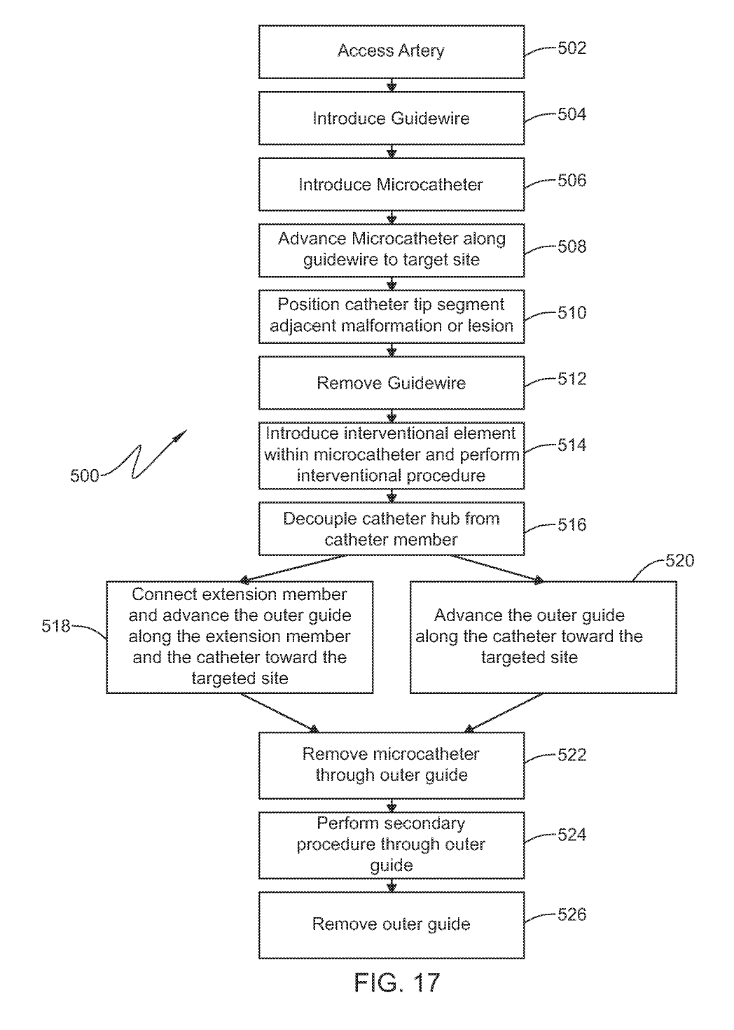

The use of the system in performing an intravascular procedure will now be discussed. Although the system may be used in a number of intravascular procedures, the following discussion will focus on the use in a neurovascular interventional procedure. In accordance with one exemplary procedure 500 detailed in the flow chart of FIG. 17, the arterial tree, e.g., the femoral artery, is accessed (STEP 502) by introducing a hollow needle into the femoral artery adjacent the groin area via a percutaneous procedure. The guidewire is introduced within the needle and advanced to a location proximate a targeted site, e.g., the aortic arch (STEP 504). The microcatheter 10 is then introduced and advanced along the guidewire 200 (STEP 506) to position the catheter tip segment 38 adjacent the aortic arch. The microcatheter 10 is navigated through the aortic arch, then into one of the carotid arteries or vertebral arteries, and then into the neurovasculature (STEP 508). The particular construction of the microcatheter 10 discussed hereinabove enables the microcatheter 10 to follow the tortuous path within the vasculature, e.g., arching over the aortic arch and turning into a coronary artery to reach the distal portion of the coronary arteries, or turning into the carotid arteries or the vertebral arteries, into the Circle of Willis and into the cerebral arteries without the use of an outer guide or support. In particular, the microcatheter 10 has sufficient lateral support to enable orientation of the catheter tip segment 38 of the microcatheter 10 in alignment with the selected carotid or vertebral artery from a remote location (e.g., by manipulation of the catheter hub 12 outside the body) while exhibiting adequate flexibility enabling the microcatheter 10 to follow these highly tortuous paths for access for the targeted vessel. The relatively soft characteristic of the catheter tip segment 38 minimizes trauma to the vasculature.

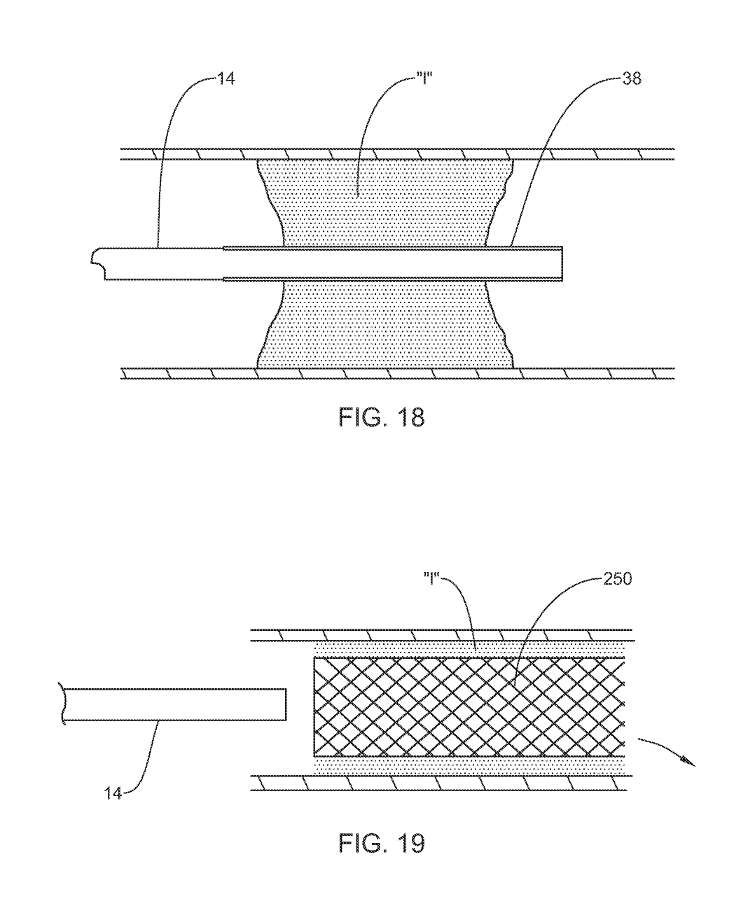

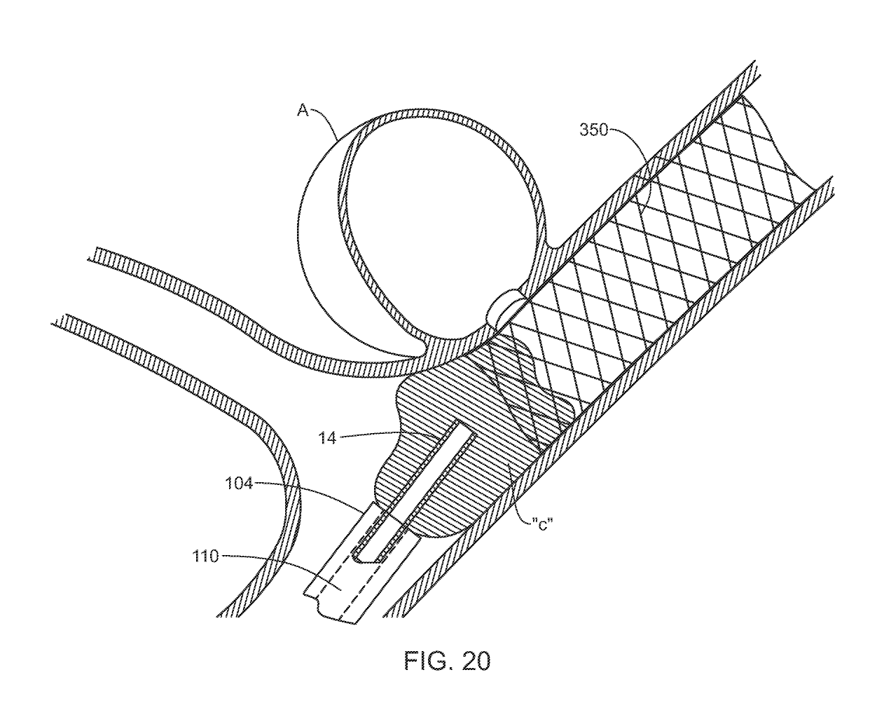

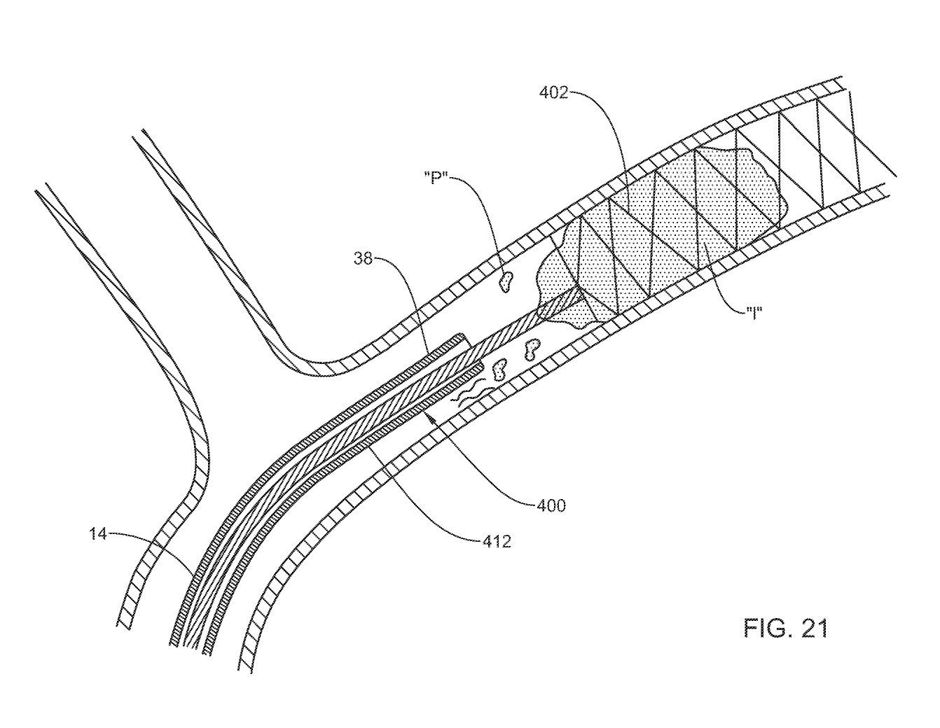

Once within the targeted neurovasculature, the catheter tip segment 38 of the microcatheter 10 is positioned adjacent the malformation such as, e.g., an aneurysm, clot, stenotic region or the like. (STEP 510) In the case of the lesion, the catheter tip segment 38 may be advanced through the lesion as depicted in FIG. 18. The guidewire 200 may be removed from the microcatheter 10 (STEP 512). Thereafter, any of the interventional devices described hereinabove in connection with FIGS. 12-16 may be advanced through the microcatheter 100 to treat the lesion or malformation (STEP 514). FIG. 19 illustrates deployment of the intravascular stent 250 within the lesion “l” thereby re-establishing blood flow through the neurovasculature. FIG. 20 illustrates deployment of the flow diverter 350 across an arteriovenous malformation (AVM) or an aneurysm “A.” FIG. 21 illustrates deployment of the SOLITAIRE™ flow restoration device 400 to capture the occlusion or lesion “l”.

Once flow is restored in the vessel, the catheter hub 12 of the microcatheter 10 is decoupled from the catheter member 14 (STEP 516). In the event the extension member 50 is required due to the length of the catheter member 14 discussed hereinabove, the extension member 50 may be coupled to the proximal end of the catheter member 14 and the outer guide 100 advanced along the extension member 50 and the catheter member 14 toward the targeted site. (STEP 518) In the event no extension member 50 is required, the outer guide 100 is advanced along the outer surface of the catheter member 14 of the microcatheter 10 to the targeted site. (STEP 520) The guide member 104 of the outer guide 100 is advanced to a location proximate the malformation or lesion. If the outer guide 100 incorporates a balloon 114, the balloon 114 may be expanded to secure the outer guide 100 within the vasculature. During or subsequent to the procedure, a contrast agent “c” may be delivered through the outer guide 100 to confirm that the interventional treatment was successful in, e.g., restoring blood flow and/or removing the lesion. In the event the interventional device is to be retrieved, the device, e.g., SOLITAIRE™ flow restoration device 400, may be removed through the microcatheter 10 or the outer guide 100 by pulling back on the deployment/retrieval rod or member 412. During use of any of the aforementioned procedures, the outer guide 100 may be used to aspirate material. Subsequent to performing the interventional treatment, the microcatheter 10 may be removed through the outer guide 100 (STEP 522). In the alternative, the microcatheter 10 may be removed simultaneously with the interventional element or device. The outer guide 100 may be removed. In the alternative, the outer guide 100 may remain within the vasculature, and additional interventional treatment elements introduced within the outer guide 100 to perform subsequent procedures (STEP 524) with any of the interventional elements discussed in connection with FIGS. 12-16, and/or introducing embolics, the contrast agent or the like. The outer guide 100 is then removed. (STEP 526)

As discussed hereinabove, the microcatheter 10 is capable of accessing the targeted neurovascular space without the need or use of an outer guide catheter or support which is typically required to cross over the aortic arch and reach into the cerebral arteries in conventional neurovascular procedures. Thus, removing the necessity of inserting an outer guide through the groin and advancing the guide catheter through, e.g., the aortic arch, will substantially reduce the time required in performing the interventional procedure and restoring flow in the neurovascular vessel. This in conjunction with the use of, e.g., the SOLITAIRE™ flow restoration device 400, will provide substantial benefits with regard to efficacy and speed, resulting in improved clinical outcomes.

Although the aforementioned steps have been described or listed in a particular order, the order of such steps may be changed unless otherwise specified or unless doing so would render the method or process unworkable for its intended purpose. For example, the interventional procedure (STEP 514) may be performed through the microcatheter 10 subsequent to removal of the catheter hub (STEP 516) and introduction of the outer guide 100 (STEP 518 or STEP 520).

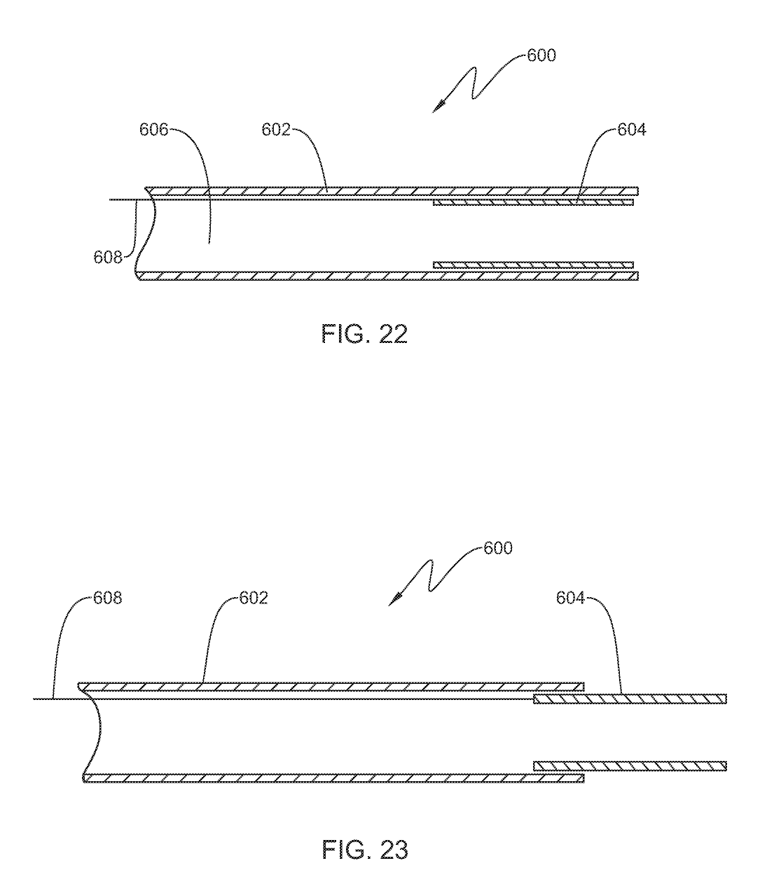

FIGS. 22-23 illustrates an alternate embodiment of the microcatheter. In accordance with this embodiment, the microcatheter 600 includes a main catheter segment 602 and a retractable catheter tip segment 604. The main catheter segment 602 and the retractable catheter tip segment 604 define a lumen therethrough. The catheter tip segment 604 is soft relative to the main body segment 602. The catheter tip segment 604 is selectively movable between a first position in which the catheter tip segment 604 is at least partially or fully disposed in the main catheter segment 602 as depicted in FIG. 21 and a second position in which the catheter tip segment 604 is at least partially or fully disposed within the main catheter segment 602 as depicted in FIG. 23. Any suitable mechanism for causing translation of the catheter tip segment 604 relative to the main catheter segment 602 are envisioned including, e.g., a drive rod 606 extending through the main catheter segment 602 and connected to the catheter tip segment 604 at the distal end of the rod and connected to an actuator or handle at the proximal end of the rod. In use, the catheter tip segment 604 is in the second deployed position exposed from the main catheter segment 602 as the microcatheter 600 is advanced through the vasculature. Upon reaching the lesion, the catheter tip segment 604 is retracted within the main catheter segment 602. In this position, the more rigid main catheter segment 602 may engage and pass through the lesion. This feature may prove advantageous when, e.g., attempting to pass through a more chronic harder lesion. Once penetration is achieved, the catheter tip segment 604 may be retracted to the first position and the interventional procedure performed as discussed hereinabove.

It is to be appreciated that the disclosure has been described hereinabove with reference to certain examples or embodiments of the disclosure but that various additions, deletions, alterations and modifications may be made to those examples and embodiments without departing from the intended spirit and scope of the disclosure. For example, any element or attribute of one embodiment or example may be incorporated into or used with another embodiment or example, unless otherwise specified to do so would render the embodiment or example unsuitable for its intended use. All reasonable additions, deletions, modifications and alterations are to be considered equivalents of the described examples and embodiments and are to be included within the scope of the following claims.

What is claimed is:

1. A system for use in a vascular procedure, which comprises:
a guidewire dimensioned to remotely access a neurovascular space;
a microcatheter including:
a catheter member defining a longitudinal axis and having a longitudinal length and with proximal and distal ends; and
a catheter hub connected to the proximal end of the catheter member, the catheter hub dimensioned and adapted to be selectively released from the catheter member;
an interventional treatment element for passage within the catheter member of the microcatheter, the interventional treatment element adapted to perform treatment on the lesion within the vasculature.
an outer guide positionable over the catheter member of the microcatheter upon removal of the catheter hub, the outer guide advanceable over the catheter member to a location proximate the lesion.

2. The system according to claim 1 wherein the catheter hub is connected to the proximal end of the catheter member through one of thread means, a bayonet coupling, a snap fit mechanism, an interference fit, an adhesive or a chemical bond.

3. The system according to claim 1 wherein the interventional treatment element is dimensioned to be introduced within the catheter member of the microcatheter upon removal of the guidewire, the interventional treatment element being selected from the group consisting of a stent, a coil, a flow diverter, a flow restoration element, a thrombectomy element, a retrieval element, an aspirator and a snare.

4. The system according to claim 3 wherein the outer guide is a balloon catheter, the balloon catheter dimensioned to receive the interventional treatment device during withdrawal thereof subsequent to performing the treatment on the lesion.

5. The system according to claim 4 wherein the balloon catheter includes an aspirator.

6. The system according to claim 1 wherein the interventional treatment element is one of an embolic solution or glue.

7. The system according to claim 1 including an extension member connectable to the proximal end of the catheter member upon removal of the catheter hub, the outer guide being advanceable over the extension member and the catheter member of the microcatheter.

8. The system according to claim 7 wherein the extension member and the proximal end of the catheter member include corresponding structure to connect the extension member to the catheter member.

9. The system according to claim 1 wherein the catheter member of the microcatheter includes a catheter tip segment, the catheter tip segment comprising a soft material relative to a neighboring segment of the catheter member proximal of the leading tip segment.

10. The system according to claim 9 wherein the catheter tip segment of the catheter member is in telescoping relation with respect to at least the neighboring segment of the catheter member adjacent the catheter tip segment, the catheter tip segment adapted for longitudinal movement between a first retracted position at least partially enclosed within the neighboring segment of the catheter member and a second extended position exposed beyond the neighboring segment.

11. The system according to claim 9 including a radiopaque marker adjacent the catheter tip segment of the catheter member of the microcatheter.

12. The system according to claim 1 wherein the catheter member of the microcatheter includes one of a hypotube segment or reinforced polymer tube segment disposed at least adjacent the proximal end of the catheter member.

13. The system according to claim 12 wherein the catheter member of the microcatheter includes one of a braided segment disposed at least adjacent the proximal end of the catheter member.

14. A method for performing a neurovascular procedure, comprising:
accessing a treatment site within a neurovascular space with a guidewire:
advancing a microcatheter over the guidewire to traverse remote locations in the neurovasculature to access the treatment site and penetrate a lesion located at the treatment site without the assistance of an independent outer guide support positioned about the microcatheter;
removing the guidewire from the microcatheter;
introducing an interventional treatment element through the microcatheter to a location adjacent the lesion within the neurovasculature;
treating the lesion with the interventional treatment element;
removing a catheter hub member from the microcatheter leaving an catheter member of the microcatheter within the neurovasculature;
positioning an outer guide over the catheter member of the microcatheter and advancing the outer guide to a location proximate the treatment site; and
withdrawing the microcatheter through the outer guide.

15. The method according to claim 14 wherein treating the lesion includes deploying a flow restoration device within the lesion to restore flow through the adjacent neurovasculature.

16. The method according to claim 15 including removing at least a portion of the lesion from the neurovasculature while withdrawing the flow restoration device through the guide catheter.

17. The method according to claim 16 including expanding a balloon member of the outer guide to secure the outer guide within the neurovasculature and then withdrawing the interventional treatment device through the outer guide and supplying aspiration to the outer guide.

18. The method according to claim 14 wherein withdrawing the microcatheter and the interventional treatment device is performed simultaneously.

19. The system according to claim 14 wherein treating the lesion includes delivering the treatment element, the treatment element selected from the group consisting of a stent, a coil, an embolic solution, glue, a flow diverter, a flow restoration element, a thrombectomy element, a retrieval element, an aspirator and a snare.

20. The system according to claim 14 wherein advancing the microcatheter includes penetrating the lesion with a catheter tip segment of the microcatheter, the catheter tip segment being relatively soft relative to a neighboring segment neighboring the catheter tip segment.

21. The system according to claim 14 including introducing a second interventional element within the outer guide subsequent to withdrawing the microcatheter.
